(19) United States
(12) Patent Application Publication
(10) Pub. No.: US 2016/0136350 A1
(43) Pub. Date: May 19, 2016

(54) IRRIGATOR FOR WASHING WHILE SURGERY OPERATION

(71) Applicant: Caleb Co., Ltd., Gangwon-do (KR)

(72) Inventor: Kyung-kil Yoo, Seoul (KR)

(73) Assignee: Caleb Co., Ltd., Gangwon-do (KR)

(21) Appl. No.: 14/937,879

(22) Filed: Nov. 11, 2015

(30) Foreign Application Priority Data
Nov. 13, 2014 (KR) 10-2014-0158356

(51) Int. Cl. A61M 3/02; A61B 17/88

(52) U.S. Cl. CPC A61M 3/0279; A61B 17/88; A61B 2217/007

(57) ABSTRACT

A surgical irrigator is disclosed. The surgical irrigator includes a body unit including an engagement module, and a spray unit engaged with the body unit. The engagement module includes a ring-type body, and an engagement unit formed on an inner circumferential surface of the body and configured to fix the spray unit in engagement with the spray unit.

### CROSS-REFERENCE TO RELATED PATENT APPLICATION

This application claims the benefit of Korean Patent Application No. 10-2014-0158356, filed on Nov. 13, 2014, which is hereby incorporated by reference as if fully set forth herein.

### BACKGROUND OF THE INVENTION

1. Field of the Invention

The present invention relates to a surgical irrigator for spraying an aqueous liquid onto an affected part during a surgical operation.

2. Discussion of the Related Art

In total joint replacement, the surface of a joint bone needs partial removal and thus the removed bone and blood should be washed out with a cleansing solution. The cleansing solution is a saline solution and an irrigator is used to spray the cleansing solution during the washing.

Total joint replacement is a surgical procedure performed on patients with degenerative arthritis, avascular necrosis with the femoral head, rheumatoid arthritis, tuberculous or inflammatory arthritis, and traumatic arthritis in any joint such as the hip, knee, shoulder, or elbow, when the patients are not treated with medication, physical therapy, and other conservative management. Total joint replacement seeks to relieve pain and restore normal motion for the patients.

As is implied from the appellation, total joint replacement seems to be a surgery in which a bone of a patient is completely cut out and an artificial bone is implanted. However, this is a wrong operation. In fact, total joint replacement is a surgical procedure in which the surface of a diseased joint bone is partially removed and covered with a thin film of a special metal and then a special plastic or ceramic is inserted between the thin film-covered bone and a bone to thereby enable the joint to make a smoother motion, like a dental procedure in which a decayed part is removed from a tooth having a cavity and the cavity is capped with gold or platinum.

A conventional irrigator includes a body with a cleansing solution spray nozzle at its one end and a space in its inside. The body is formed of a flexible material so as to be readily deformed just by a force applied by a hand.

To inject a cleansing solution into the internal space of the body, air is discharged from the body by applying a force to the body. In this state, the cleansing solution spray nozzle is immersed in a cleansing solution reservoir and the cleansing solution is sucked into the body by releasing the force from the body.

With the cleansing solution injected into the internal space of the body, the cleansing solution spray nozzle is directed to an affected part on which a surgical operation will be performed. Subsequently, when a force is applied to the body, the cleansing solution from the body is sprayed onto the affected part and the affected part is cleansed with the sprayed cleansing solution.

Because this irrigator is operated in contact with an affected part of a patient, it is very important to keep the irrigator clean. Thus, a spray unit should be replaced or detached from the irrigator, for cleaning. However, it is difficult to remove the spray unit from the body in the conventional irrigator.

### SUMMARY OF THE INVENTION

Accordingly, the present invention is directed to a surgical irrigator that substantially obviates one or more problems due to limitations and disadvantages of the related art.

An object of the present invention is to provide a surgical irrigator configured to allow easy removal/engagement of a spray unit from/with a body unit.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, a surgical irrigator includes a body unit including an engagement module, and a spray unit engaged with the body unit. The engagement module includes a ring-type body, and an engagement unit formed on an inner circumferential surface of the body and configured to fix the spray unit in engagement with the spray unit.

The engagement unit may include a first engagement unit and a second engagement unit that are disposed symmetrically with respect to a hole of the ring-type body.

The engagement unit may further include a third engagement unit and a fourth engagement unit that are disposed symmetrically with respect to the hole of the ring-type body, between the first engagement unit and the second engagement unit.

Each of the first and second engagement units may include a catching member apart from the body, and a connection member configured to connect the catching member to the inner circumferential surface of the body.

The catching member may be shaped into a sphere.

The connection member may include a first member and a second member that are disposed symmetrically with respect to the catching member.

The first and second members may be elastic members.

The spray unit may include an outer body contacting a housing of the body unit, and an inner body inserted into the hole.

The inner body may include a mounting groove formed on an outer circumferential surface of the inner body and configured to allow the catching member to be mounted thereon.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a perspective view of a surgical irrigator according to an embodiment of the present invention;

FIG. 2 is a sectional view of the surgical irrigator illustrated in FIG. 1;

FIG. 3 is an enlarged conceptual view of an engagement unit illustrated in FIG. 2;

FIG. 4 is a conceptual view of one end of a spray unit;

FIG. 5A is a conceptual view of an engagement module according to an embodiment of the present invention; and

FIG. 5B is a conceptual view of the engagement module illustrated in FIG. 5A, viewed from a different direction.

### DETAILED DESCRIPTION OF THE INVENTION

A surgical irrigator to which the present invention relates to will be described in detail with reference to the accompanying drawings. Like or similar reference numerals denote the same or similar components even in different embodiments. Once the same or similar components are described, the description is applied to them across the disclosure. Singular terms are intended to include plural forms, unless the context makes it clear that plural forms are not intended.

Terms ‘module’ and ‘unit’ used to name components are allocated or used interchangeably only in consideration of the easiness of writing the disclosure, without any distinguishing meaning or role.

As used in the description of the present invention, terms such as ‘first’ or ‘second’ may be used to describe various components, but do not limit such components. Such terms may be used to distinguish one component from another. For example, without departing from the scope of the present invention, a first component may be called a second component, and likewise, a second component may be called a first component.

If a component is said to be ‘connected with’ or ‘connected to’ another component, the component may be directly connected with, or connected to, the other component, or another component may exist in between. On the other hand, if a component is said to be ‘directly connected with’ or ‘directly connected to’ another component, it should be understood that no components exist in between.

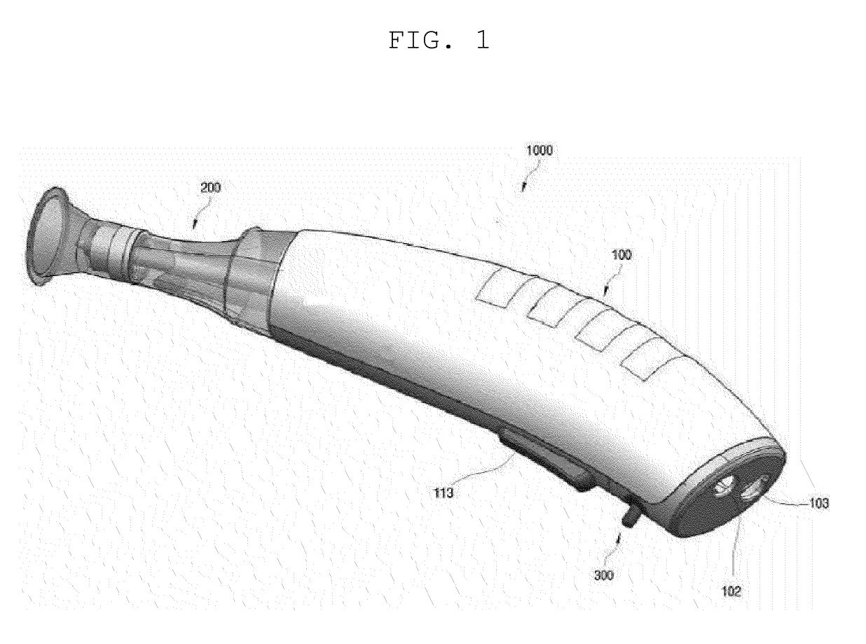

FIG. 1 is a perspective view of a surgical irrigator 1000 according to an embodiment of the present invention.

Referring to FIG. 1, the surgical irrigator 1000 includes a body unit 100 and a spray unit 200.

The spray unit 200 is detachably engaged with one portion of the body unit 100. The body unit 100 may include a switch 113 and a control module 300 configured to control a sprayed amount of water.

According to an embodiment of the present invention, the body unit 100 may include a first hole 102 and a second hole 103.

A pipe connected to a cleansing solution reservoir (not shown) outside the surgical irrigator 1000 may pass through the first hole 102. In general, a saline solution is used as a cleansing solution and thus the cleansing solution reservoir contains the saline solution. The cleansing solution reservoir may be a saline solution bottle called a fluid bottle.

A vacuum suction pipe connected to a vacuum pump (not shown) may pass through the second hole 103. The vacuum pump may be connected to the surgical irrigator 1000 of the present invention to suck out the cleansing solution sprayed onto a surgical site and blood and bone pieces from the surgical site by vacuum. The vacuum pump may be connected to the surgical irrigator 1000 through the second hole 103.

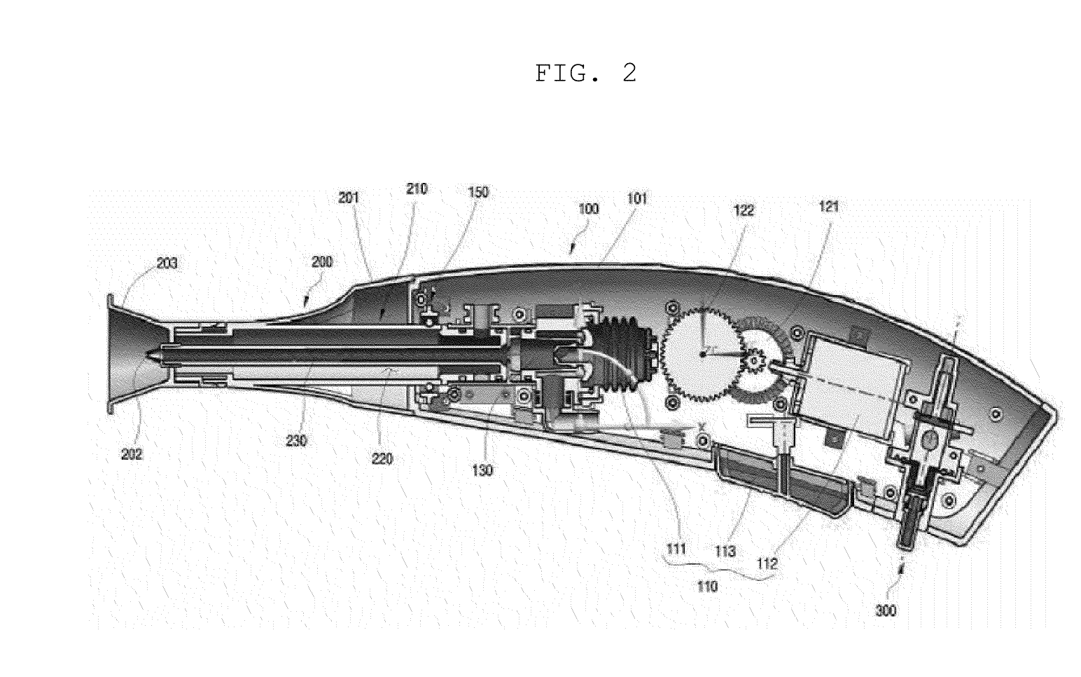

FIG. 2 is a sectional view of the surgical irrigator 1000 illustrated in FIG. 1.

Referring to FIG. 2, the surgical irrigator 1000 includes the body unit 100 and the spray unit 200.

The body unit 100 includes a housing 101 and various parts accommodated in the housing 101.

A cleansing solution supply module 110, the cleansing solution control module 300, and a driving force transfer module may be accommodated in the housing 101.

The cleansing solution supply module 110 may include an extension member 111, a driving motor 112, and the switch 113. The cleansing solution supply module 110 supplies the cleansing solution from the cleansing solution reservoir to the surgical irrigator 1000.

According to an embodiment of the present invention, the extension member 111 takes the form of an extendable bellow and is extended and shrunken by operations of the driving motor 112. The number of revolutions of the driving motor 112 is controllable by a user manipulation.

The driving force transfer module is configured to transfer a driving force of the driving motor 112 to the extension member 111. The driving force transfer module may include a first gear 121, a second gear 122, and a cam (not shown).

The first gear 121 may be a bevel gear that transfers rotation of an axis of the driving motor 112 to the second gear 122. The second gear 122 may be coupled to the cam (not shown). The cam is coupled to one end of the extension member 111 and transfers movement of the second gear 122 to the extension member 111. In other words, a rotation force of the driving motor 112 is transferred to the extension member 111 by means of the first gear 121, the second gear 122, and the cam and the extension member 111 is extended or shrunken by the transferred force.

The switch 113 controls a current applied to the driving motor 112. The switch 113 is a variable switch and may control the magnitude of the current applied to the driving motor 112. If the magnitude of the current applied to the driving motor 112 is changed, the number of revolutions of the driving motor 112 is also changed, thereby controlling an amount of the cleansing solution sprayed through the surgical irrigator 1000.

A cleansing solution chamber 130 serves as a temporary reservoir that temporarily contains the cleansing solution before it is sprayed onto a surgical site. The cleansing solution chamber 130 is connected to a cleansing solution spray tube 230. As the extension member 111 is extended and shrunken, a pressure is applied to the cleansing solution chamber 130 and the cleansing solution is sprayed through the spray tube 230 and then a spray nozzle 202.

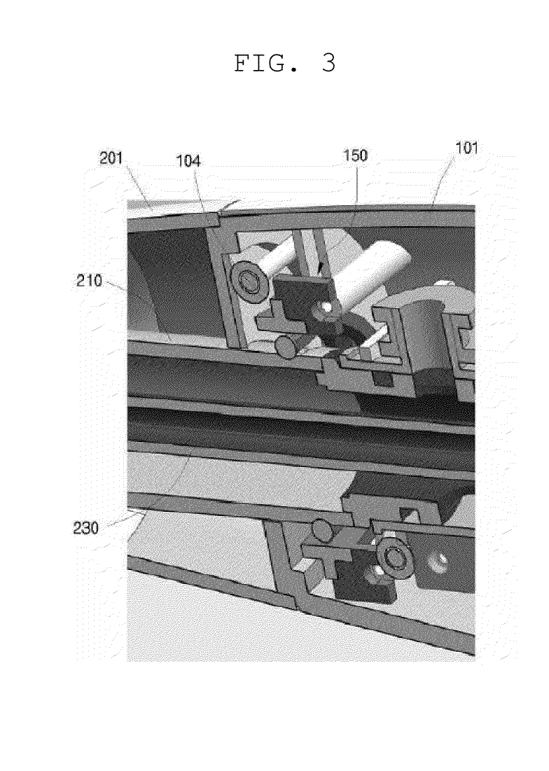

FIG. 3 is an enlarged conceptual view of an engagement unit illustrated in FIG. 2. Specifically, an engagement state between an engagement module 150 and the spray unit 200 is illustrated in FIG. 3.

Referring to FIG. 3, the housing 101 of the body unit 100 is fixedly engaged with an outer body 201 of the spray unit 200 and an inner body 210 of the spray unit 200 is fixedly engaged with the engagement module 150.

A protrusion 104 may be formed inside the housing 101, in order to fix the engagement module 150.

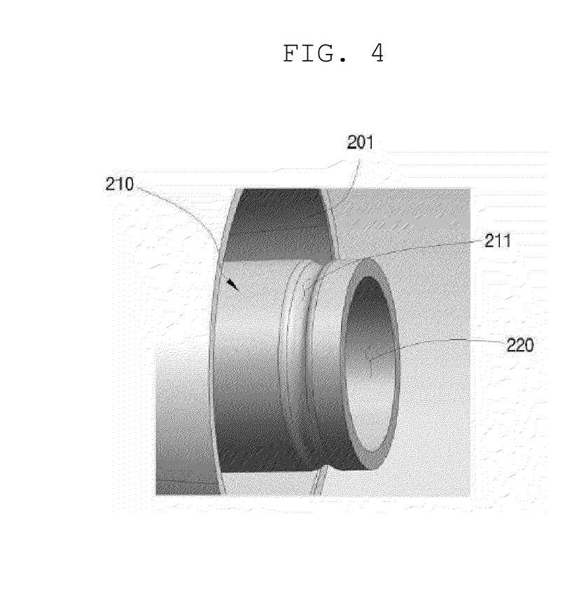

FIG. 4 is a conceptual view of one end of the spray unit 200.

Referring to FIG. 4, the spray unit 200 includes the outer body 201 and the inner body 210. A hole 220 may be formed in the inner body 210, in which the cleansing solution spray tube 230 is inserted.

A mounting groove 211 may be formed around the outer circumference of the inner body 210. Referring to FIGS. 3 and 4, a catching member is mounted in the mounting groove 211, thereby preventing free slip-off of the spray unit 200 from the body unit 100.

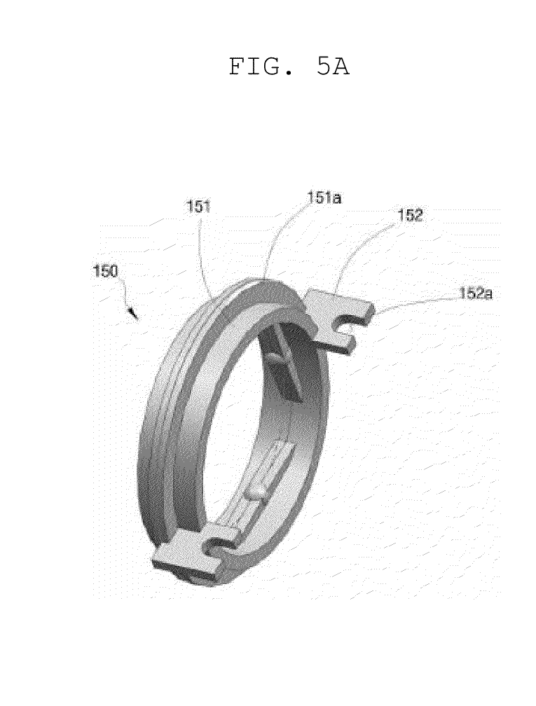

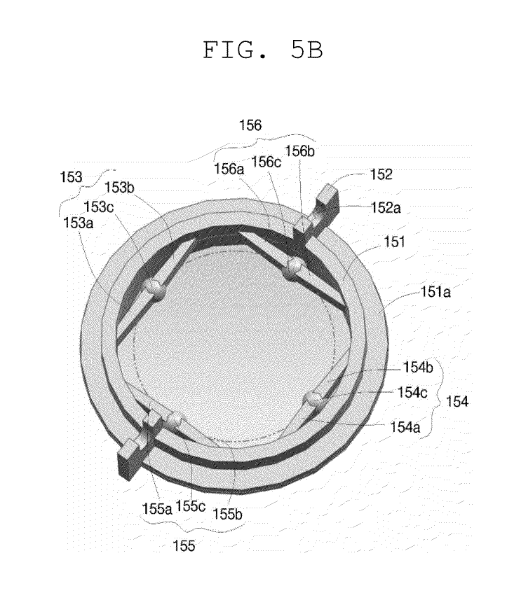

FIG. 5A is a conceptual view of the engagement module 150 according to an embodiment of the present invention, and FIG. 5B is a conceptual view of the engagement module 150 illustrated in FIG. 5A, viewed from a different direction.

Referring to FIGS. 5A and 5B, the engagement module 150 includes a ring-type body 151 and an engagement unit formed on an inner circumferential surface of the body 151, for fixing the spray unit 200 in engagement with the spray unit 200. More specifically, the engagement unit prevents free slip-off of the spray unit 200 by fixing the inner body of the spray unit 200.

The engagement unit may include a first engagement unit 153 and a second engagement unit 154 which are symmetrical with respect to a hole at the center of the body 151.

The first engagement unit 153 includes a catching member 153c apart from the inner circumferential surface of the body 151, and a first member 153a and a second member 153b that connect the catching member 153c to the body 151. The catching member 153c may be shaped into a sphere. The first and second members 153a and 153b may be elastic. When the spray unit 200 is engaged with the body unit 100, the catching member 153c is pushed toward the inner circumferential surface of the body 151 and then mounted in the mounting groove 211 formed on the inner body 210 of the spray unit 200. Because the first and second members 153a and 153b are elastic, the catching member 153c may be mounted in the mounting groove 211 by a restoring force after the catching member 153c is pushed and then inserted during insertion of the spray unit 200. As the catching member 153c is caught in the mounting groove 211, the spray unit 200 may not be slipped off freely. Further, the spray unit 200 may be readily removed from the body unit 100 by pulling the spray unit 200 with a force equal to or stronger than a predetermined value, since the first and second members 153a and 153b have an elastic force.

Like the first engagement unit 153, the second engagement unit 154 includes a catching member 154c and members 154a and 154b that connect the catching member 154c to the inner circumferential surface of the body 151.

According to another embodiment of the present invention, the engagement unit may further include third and fourth engagement units 155 and 156 which are symmetrical with respect to the hole at the center of the body 151, between the first and second engagement units 153 and 154.

The third and fourth engagement units 155 and 156 may have the same configuration as the first and second engagement units 153 and 154. In other words, the third engagement unit 155 may include a catching member 155c and members 155a and 155b that connect the catching member 155c to the body 151, and the fourth engagement unit 156 may include a catching member 156c and members 156a and 156b that connect the catching member 156c to the body 151.

An extension unit 151a may be formed in the body 151 of the engagement module 150 so that the body 151 may be caught over the protrusion 104 inside the housing 101.

A fixing plate 152 may be formed on an outer circumferential surface of the engagement module 150 to fix the body 151 to the housing 101. A screw hole 152a may be formed into the fixing plate 152. Referring to FIG. 3, the engagement module 150 may be assembled more firmly by engaging the fixing plate 152 with the protrusion 104 and inserting a screw into the screw hole 152a.

As is apparent from the foregoing description, the surgical irrigator according to at least one of the above-described embodiments of the present invention includes an engagement module engaged with a spray unit and the engagement module includes a catching member fit in a groove of the spray unit and an elastic member engaged with the catching member. Therefore, a user can readily attach/remove the spray unit after using the surgical irrigator, which makes a surgical operation more efficient and more clean.

The above-described surgical irrigator 1000 is not limited to the configurations and methods of the foregoing embodiments of the present invention. Rather, many variations and modifications may be made to the embodiments by selectively implementing the whole or part of each embodiment.

Those skilled in the art will appreciate that the present invention may be carried out in other specific ways than those set forth herein without departing from the spirit and essential characteristics of the present invention. The above embodiments are therefore to be construed in all aspects as illustrative and not restrictive. The scope of the invention should be determined by the appended claims and their legal equivalents, not by the above description, and all changes coming within the meaning and equivalency range of the appended claims are intended to be embraced therein.

What is claimed is:

1. A surgical irrigator comprising:
a body unit including an engagement module; and
a spray unit engaged with the body unit,
wherein the engagement module comprises:
a ring-type body; and
an engagement unit formed on an inner circumferential surface of the body and configured to fix the spray unit in engagement with the spray unit.

2. The surgical irrigator according to claim 1, wherein the engagement unit comprises a first engagement unit and a second engagement unit that are disposed symmetrically with respect to a hole of the ring-type body.

3. The surgical irrigator according to claim 2, wherein the engagement unit further comprises a third engagement unit and a fourth engagement unit that are disposed symmetrically with respect to the hole of the ring-type body, between the first engagement unit and the second engagement unit.

4. The surgical irrigator according to claim 2, wherein each of the first and second engagement units comprises:
a catching member apart from the body; and
a connection member configured to connect the catching member to the inner circumferential surface of the body.

5. The surgical irrigator according to claim 4, wherein the catching member is shaped into a sphere.

6. The surgical irrigator according to claim 4, wherein the connection member includes a first member and a second member that are disposed symmetrically with respect to the catching member.

7. The surgical irrigator according to claim 6, wherein the first and second members are elastic members.

8. The surgical irrigator according to claim 4, wherein the spray unit comprises:
an outer body contacting a housing of the body unit; and
an inner body inserted into the hole.

9. The surgical irrigator according to claim 8, wherein the inner body includes a mounting groove formed on an outer circumferential surface of the inner body and configured to allow the catching member to be mounted thereon.
